SEQUENCE LISTING (9 sequences)

SEQ ID NO: 1 - DNA, 1456 bp, Clostridium sp.
  misc_feature 1..1456: note = 16s rRNA gene of Clostridium bolteae
  misc_feature 1..1456: note = Inventor: Ito Akihiro; Abe Takaaki; Katayama Hiromichi
  source 1..1456: mol_type = genomic DNA
gatgaacgctggcggcgtgcctaacacatgcaagtcgaacgaagcaattaaaatgaagttttcggatggatttttaattgactgagtggcggacgggtgagtaacgcgtggataacctgcctcacactgggggataacagttagaaatgactgctaataccgcataagcgcacagtgccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccgcctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaaggcagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtg

SEQ ID NO: 2 - DNA, 1438 bp, Eubacterium sp.
  misc_feature 1..1438: note = 16s rRNA gene of Eubacterium coprostanoligenes iPAC001050_s j
  source 1..1438: mol_type = genomic DNA
gacgaacgctggcggcgtgcctaacacatgcaagtcgaacggacgaggaggagcttgcttctccaagtcagtggcggacgggtgagtaacgcgtgagtaacctgcccttcagagggggataacgtctggaaacggacgctaataccgcatgacatatttccatcgcatggtggagatatcaaaggagcaatccgctgaaggatggactcgcgtccgattagatagttggcggggtaacggcccaccaagtcgacgatcggtagccggactgagaggttgaacggccacattgggactgagacacggcccagactcctacgggaggcagcagtgggggatattgcacaatgggcgaaagcctgatgcagcaacgccgcgtgagggaagacggttttcggattgtaaacctctgttctaagtgacgaacaaatgacggtagcttaggagaaagccccggctaactacgtgccagcagccgcggtaatacgtagggggcaagcgttgtccggaattactgggtgtaaagggagcgcaggcggagaagcaagtcagtggtgaaaacgatgggcttaactcatcgactgccattgaaactgtttcccttgagtgaagtagaggcaggcggaattccgagtgtagcggtgaaatgcgtagatattcggaggaacaccagtggcgaaggcggcctgctgggctttaactgacgctgaggctcgaaagtgtggggagcaaacaggattagataccctggtagtccacactgtaaacgatgattgctaggtgtggggggtctgaccccttccgtgccggagttaacgcaataagcaatccacctggggagtacggccgcaaggttgaaactcaaaggaattgacgggggcccgcacaagcagtggattatgtggtttaattcgacgcaacgcgaagaaccttaccaggtcttgacatccaacgaatccttaagagattagggagtgcctttcggggaacgttgagacaggtggtgcatggttgtcgtcagctcgtgtcgtgagatgttgggttaagtcccgcaacgagcgcaacccctgccattagttgctacgcaagagcactctaatgggaccgctaccgacaaggtggaggaaggtggggacgacgtcaaatcatcatgccccttatgacctgggctacacacgtaatacaatggccgttaacaacgggaggcaaaaccgcgaggtggagcaaaaccctaaaaacggtcccagttcggatcgcaggctgcaacccgcctgcgtgaagttggaattgctagtaatcgcggatcagcatgccgcggtgaatacgttcccgggccttgtacacaccgcccgtcacaccatgggagtttgtaacacccgaagtcagtagtctaaccgcaagggggacgctgccgaaggtgggataagcgactggggtg

SEQ ID NO: 3 - DNA, 1452 bp, unidentified
  misc_feature 1..1452: note = 16s rRNA gene of FCEY_s
  source 1..1452: mol_type = genomic DNA
gatgaacgctggcggcgtgcctaacacatgcaagtcgagcgaagcggttttaaggaagttttcggatggaattaaaactgactgagcggcggacgggtgagtaacgcgtgggtaacctgcctcatacagggggataacagttagaaatggctgctaataccgcataagcacacagcttcgcatggagcagtgtgaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcggagcaagtctgaagtgaaagcccggggctcaaccccgggactgctttggaaactgttctgctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgaatgctaggtgttggagggcaaagcccttcggtgccgccgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggacagtaatgtgtcctttccttcgggacaggggagacaggtggtgcatggttgtcgtcagctcgtgtcgtgagatgttgggttaagtcccgcaacgagcgcaacccttatccttagtagccagcagtaagatgggcactctagggagactgccagggataacctggaggaaggtggggatgacgtcaaatcatcatgccccttatgatttgggctacacacgtgctacaatggcgtaaacaaagagaagcgagcctgcgagggggagcgaatctcaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgactcaaccgcaaggagagagctgccgaaggcggggcaggtaactggggtg

SEQ ID NO: 4 - DNA, 1454 bp, unidentified
  misc_feature 1..1454: note = 16s rRNA gene of Anaerotignum lactatifermentans
  source 1..1454: mol_type = genomic DNA
gatgaacgctggcggcgtgcttaacacatgcaagtcgaacgaagatttagaatgagagcttcggcaggatttttatctatcttagtggcggacgggtgagtaacgtgtgggcaacctgccctgtactggggaataatcattggaaacgatgactaataccgcatgtggtcctcggaaggcatcttctgaggaagaaaggatttattcggtacaggatgggcccgcatctgattagctagttggtgagataacagcccaccaaggcgacgatcagtagccgacctgagagggtgatcggccacattgggactgagacacggcccaaactcctacgggaggcagcagtggggaatattgcacaatgggcgaaagcctgatgcagcaacgccgcgtgaaggatgaagggtttcggctcgtaaacttctatcaatagggaagaaataaatgacggtacctaaataagaagccccggctaactacgtgccagcagccgcggtaatacgtagggggcaagcgttatccggaattactgggtgtaaagggagcgtaggcggcatggtaagccagatgtgaaagccttgggcttaacccgaggattgcatttggaactatcaagctagagtacaggagaggaaagcggaattcctagtgtagcggtgaaatgcgtagatattaggaagaacaccagtggcgaaggcggctttctggactgaaactgacgctgaggctcgaaagcgtggggagcaaacaggattagataccctggtagtccacgccgtaaacgatgagtgctaggtgtcggggaggaatcctcggtgccgcagctaacgcaataagcactccacctggggagtacgaccgcaaggttgaaactcaaaggaattgacgggggcccgcacaagcggtggagcatgtggtttaattcgaagcaacgcgaagaaccttaccaaggcttgacatcccgatgaccgtcctagagataggacttctcttcggagcatcggtgacaggtggtgcatggttgtcgtcagctcgtgtcgtgagatgttgggttaagtcccgcaacgagcgcaacccttatcttcagtagccatcattcagttgggcactctggagagactgccgtggataacacggaggaaggtggggatgacgtcaaatcatcatgccccttatgtcttgggctacacacgtgctacaatggctggtaacaaagtgacgcaaaacggcgacgttaagcaaatcacaaaaacccagtcccagttcggattgtagtctgcaactcgactacatgaagctggaatcgctagtaatcgcgaatcagcatgtcgcggtgaatacgttcccgggccttgtacacaccgcccgtcacaccatgggagttggaagcacccgaagtcggtgacctaaccgtaaggaaggagccgccgaaggtgaagccagtgactggggtg

SEQ ID NO: 5 - DNA, 1455 bp, unidentified
  misc_feature 1..1455: note = 16s rRNA gene of LT635549_s
  source 1..1455: mol_type = genomic DNA
gatgaacgctggcggcgtgcctaacacatgcaagtcgagcgaagcactttacttagatttcttcggaatgacgagtttagtgactgagcggcggacgggtgagtaacgcgtgggtaacctgcctcatacagggggataacagttagaaatgactgctaataccgcataagaccacagcttcgcatggagcggtggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaactgacggtacctgactaagaagcaccggctaaatacgtgccagcagccgcggtaatacgtatggtgcaagcgttatccggatttactgggtgtaaagggagcgtagacggataggcaagtctggagtgaaagcccggggctcaaccccgggactgctttggaaactgtttatctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgcccttgacatccggatgaccggtgggtaatgccaccctctcttcggagcatccgagacaggtggtgcatggttgtcgtcagctcgtgtcgtgagatgttgggttaagtcccgcaacgagcgcaacccttatctttagtagccagcattcaggatgggcactctagagagactgccagggataacctggaggaaggtggggatgacgtcaaatcatcatgccccttatgggcagggctacacacgtgctacaatggcgtaaacaaagggaagcgagtctgtgaggataagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtg

SEQ ID NO: 6 - DNA, 1450 bp, unidentified
  misc_feature 1..1450: note = 16s rRNA gene of Ruthenibacterium lactatiformans
  source 1..1450: mol_type = genomic DNA
gacgaacgctggcggcgcgcctaacacatgcaagtcgaacggagctgttttctctgaagttttcggatggaagagagttcagcttagtggcgaacgggtgagtaacacgtgagcaacctgcctttcagtgggggacaacatttggaaacgaatgctaataccgcataagaccacagtgtcgcatggcacaggggtcaaaggatttatccgctgaaagatgggctcgcgtccgattagctagatggtgaggtaacggcccaccatggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgtcccaggggacgataatgacggtaccctgggaggaagcaccggctaactacgtgccagcagccgcggtaaaacgtagggtgcaagcgttgtccggaattactgggtgtaaagggagcgcaggcggattggcaagttgggagtgaaatctatgggctcaacccataaattgctttcaaaactgtcagtcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcggatgcatacctaagagattagggaagtccttcgggacatccagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggctattaacagagagaagcgataccgcgaggtggagcaaacctcacaaaaatagtctcagttcggatcgcaggctgcaacccgcctgcgtgaagccggaattgctagtaatcgcggatcagcatgccgcggtgaatacgttcccgggccttgtacacaccgcccgtcacaccatgagagccggggggacccgaagtcggtagtctaaccgtaaggaggacgccgccgaaggtaaaactggtgattggggtg

SEQ ID NO: 7 - DNA, 1450 bp, unidentified
  misc_feature 1..1450: note = 16s rRNA gene of PAC001646_s
  source 1..1450: mol_type = genomic DNA
gacgaacgctggcggcgcgcctaacacatgcaagtcgaacggagttattttgacagaagccttcgggcagacgttgatataacttagtggcgaacgggtgagtaacacgtgagcaacctgcccttcagagggggacaacagttggaaacgactgctaataccgcataagaccacggtgtggcatcacacagaggtcaaaggagaaatccgctgaaggatgggctcgcgtccaattagctagatggtgaggtaacggcccaccatggcgacgattggtagccggactgagaggttgatcggccacattgggactgagacacggcccagactcctacgggaggcagcagtggggaatattgcacaatggaggaaactctgatgcagcgacgccgcgtgagggaagaaggtcttcggattgtaaacctctgttgtcagggacgataatgacggtacctgacgaggaagccacggctaactacgtgccagcagccgcggtaaaacgtaggtggcaagcgttgtccggaattactgggtgtaaagggagcgcaggcgggagagcaagttggaagtgaaatctgtgggctcaactcacaaattgctttcaaaactgtttttcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtggaaggattgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcccgtgcatagctaagagattagtgaagtccttcgggacacggagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggctattaacagagagaagcgaagccgcgaggtagagcaaacctcagaaaaatagtctcagttcggatcgcaggctgcaacccgcctgcgtgaagccggaattgctagtaatcgcggatcagcatgccgcggtgaatacgttcccgggccttgtacacaccgcccgtcacaccatgagagccggggggacccgaagtcggtagtctaaccgcaaggaggacgccgccgaaggtaaaactggtgattggggtg

SEQ ID NO: 8 - DNA, 1454 bp, unidentified; identical to SEQ ID NO: 4
  misc_feature 1..1454: note = 16s rRNA gene of Anaerotignum lactatifermentans
  source 1..1454: mol_type = genomic DNA

SEQ ID NO: 9 - DNA, 1431 bp, unidentified
  misc_feature 1..1431: note = 16s rRNA gene of Eggerthella lenta
  source 1..1431: mol_type = genomic DNA
gatgaacgctggcggcgtgcctaacacatgcaagtcgaacgatgaaaccgccctcgggcggacatgaagtggcgaacgggtgagtaacacgtgaccaacctgccccttgctccgggacaaccttgggaaaccgaggctaataccggatactcctcgcccccctcctggggggcccgggaaagcccagacggcaagggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggaggcgtcgaaggtgtggagggtaaggggggtg

(19) United States
(12) Patent Application Publication
(10) Pub. No.: US 2026/0286451 A1
(43) Pub. Date: Sep. 24, 2026

(54) Intestinal Bacteria Marker for Diagnosing Kidney Cancer

(71) Applicant: TOHOKU UNIVERSITY, Miyagi (JP)

(72) Inventors: Akihiro Ito, Miyagi (JP); Takaaki Abe, Miyagi (JP); Hiromichi Katayama, Miyagi (JP)

(21) Appl. No.: 19/557,401

(22) Filed: Mar. 5, 2026

Related U.S. Application Data
(63) Continuation-in-part of application No. PCT/JP2024/032287, filed on Sep. 10, 2024.

(30) Foreign Application Priority Data
Sep. 11, 2023 (JP) 2023-146726

(51) Int. Cl. C12Q 1/689

(52) U.S. Cl. CPC C12Q 1/689; C12Q 2600/112

(57) ABSTRACT

An object of the present invention is to provide an intestinal bacterial marker for kidney cancer diagnosis. Kidney cancer is determined by calculating an abundance ratio of a specific intestinal bacterium to all intestinal bacteria, and using the abundance ratio as an indicator.

### CROSS-REFERENCE TO RELATED APPLICATIONS

This application claims priority to and is a continuation-in-part of International Application No. PCT/JP2024/032287, filed Sep. 10, 2024, which claims priority to Japan Application Serial No. 2023-146726, filed Sep. 11, 2023, the entire contents of which are incorporated herein by reference.

### INCORPORATION-BY-REFERENCE OF MATERIALS FILED ON COMPACT DISC

The present application includes a Sequence Listing which has been submitted in XML format via EFS-Web and is hereby incorporated by reference in its entirety. Said XML copy, created on Jan. 21, 2026, is named HRTA1071CIP_SeqList.xml and is 28000 bytes in size.

### TECHNICAL FIELD OF THE INVENTION

The present invention relates to, for example, a method for determining kidney cancer, comprising calculating an abundance ratio of a specific intestinal bacterium to all intestinal bacteria.

### BACKGROUND OF THE INVENTION

Microbes are originally foreign matter to host organisms and can therefore be eliminated by the immune system. Nonetheless, intestinal symbionts are characterized by living symbiotically in the intestinal tract without receiving aggressive elimination by the enteric immune system. Mammals including humans are born in a germ-free condition and are then infected by various bacteria through contact with the outside world so that intestinal flora is constituted. In the human intestinal tract, approximately 1000 or more types of intestinal symbionts exist in approximately one hundred trillion or more individuals and are considered to largely influence an environment in the intestinal tract.

The relation of intestinal flora to various diseases has also been elucidated in recent years. For example, it has been reported that the amounts of specific intestinal bacteria are increased in prostate cancer patients (patent document 1). A method for evaluating a disease risk by classifying a type of intestinal flora has also been reported (patent document 2). The relation of intestinal flora to clear cell renal cell carcinoma (ccRCC) has also been reported (non-patent document 1). However, it has not been known so far that the intestinal bacterium of the present invention mentioned later is useful as an intestinal bacterial marker for kidney cancer diagnosis.

### SUMMARY OF THE INVENTION

### Object to be Solved by the Invention

An object of the present invention is to provide an intestinal bacterial marker for kidney cancer diagnosis.

### Means to Solve the Object

The present inventors have been continuing diligent studies to attain the object. During the course thereof, bacteria of five types of genera (Ruminococcus_g4, the genus Ruthenibacterium, Clostridium g24, the genus Anaerotignum, and the genus Eggerthella) have been identified, by analysis at the genus level, as intestinal bacteria, the abundance ratio of which relative to all intestinal bacteria in kidney cancer patients was confirmed to be significantly increased in comparison with the abundance ratio in non-cancer control subjects. Four types of bacteria (Clostridium bolteae, Eubacterium coprostanoligenes [PAC001050_s], a bacterium of the genus Enterocloster [FCEY_s], and Anaerotignum lactafifermentas) have been further identified by analysis at the species level. The present inventors have found that among these intestinal bacteria, a bacterium of one type of genus (the genus Anaerotignum) is useful as an intestinal bacterial marker for diagnosis of kidney cancer of stage I to II, bacteria of two types of genera (Ruminococcus g4 and the genus Ruthenibacterium) and one type of bacterium (PAC001050_s) are useful as intestinal bacterial markers for diagnoses of kidney cancer of stage III to IV, bacteria of two types of genera (Clostridium_g24 and the genus Eggerthella) and three types of bacteria (Clostridium bolteae, FCEY_s, and Anaerotignum lactafifermentas) are useful as intestinal bacterial markers for diagnoses of kidney cancer of stage I to II and kidney cancer of stage III to IV, leading to the completion of the present invention.

Specifically, the present invention is as follows.
[1] A method for determining kidney cancer, comprising the following steps (a) and (b-1):
(a) separating intestinal bacterium-derived genomic DNA from an intestinal bacterium-containing sample collected from a test subject; and
(b-1) on the basis of nucleotide sequence information on the intestinal bacterium-derived genomic DNA,
calculating, relative to all intestinal bacteria, an abundance ratio of one or more bacteria selected from the group consisting of i) a bacterium of the genus Clostridium having 16S rRNA gene having at least 90% sequence identity to the nucleotide sequence set forth in SEQ ID NO: 1, ii) a bacterium of the genus Eubacterium having 16S rRNA gene having at least 90% sequence identity to the nucleotide sequence set forth in SEQ ID NO: 2, iii) a bacterium of the genus Enterocloster having 16S rRNA gene having at least 95% sequence identity to the nucleotide sequence set forth in SEQ ID NO: 3, and iv) a bacterium of the genus Anaerotignum having 16S rRNA gene having at least 90% sequence identity to the nucleotide sequence set forth in SEQ ID NO: 4, wherein
when the abundance ratio of the bacterium i) is increased compared with that in a non-cancer control subject, the test subject is found likely to have kidney cancer of stage I to II or stage III to IV,
when the abundance ratio of the bacterium ii) is increased compared with that in a non-cancer control subject, the test subject is found likely to have kidney cancer of stage III to IV,
when the abundance ratio of the bacterium iii) is increased compared with that in a non-cancer control subject, the test subject is found likely to have kidney cancer of stage I to II or stage III to IV, and
when the abundance ratio of the bacterium iv) is increased compared with that in a non-cancer control subject, the test subject is found likely to have kidney cancer of stage I to II.
[2] The method for determining kidney cancer according to [1], wherein
the bacterium i) is Clostridium bolteae having 16S rRNA gene having at least 97% sequence identity to the nucleotide sequence set forth in SEQ ID NO: 1,
the bacterium ii) is Eubacterium coprostanoligenes having 16S rRNA gene having at least 97% sequence identity to the nucleotide sequence set forth in SEQ ID NO: 2,
the bacterium iii) is a bacterium of the genus Enterocloster having 16S rRNA gene having at least 97% sequence identity to the nucleotide sequence set forth in SEQ ID NO: 3, and
the bacterium iv) is Anaerotignum lactafifermentas having 16S rRNA gene having at least 97% sequence identity to the nucleotide sequence set forth in SEQ ID NO: 4.
[3] A method for determining kidney cancer, comprising the following steps (a) and (b-2):
(a) separating intestinal bacterium-derived genomic DNA from an intestinal bacterium-containing sample collected from a test subject; and
(b-2) on the basis of nucleotide sequence information on the intestinal bacterium-derived genomic DNA,
calculating, relative to all intestinal bacteria, an abundance ratio of one or more bacteria selected from the group consisting of a bacterium of the genus Mediterraneibacter, a bacterium of the genus Ruthenibacterium, a bacterium of the genus Enterocloster, a bacterium of the genus Anaerotignum, and a bacterium of the genus Eggerthella, wherein
when the abundance ratio of the bacterium of the genus Mediterraneibacter is increased compared with that in a non-cancer control subject or a patient with kidney cancer of stage I to II, the test subject is found likely to have kidney cancer of stage III to IV,
when the abundance ratio of the bacterium of the genus Ruthenibacterium is increased compared with that in a non-cancer control subject, the test subject is found likely to have kidney cancer of stage III to IV,
when the abundance ratio of the bacterium of the genus Enterocloster is increased compared with that in a non-cancer control subject, the test subject is found likely to have kidney cancer of stage I to II,
when the abundance ratio of the bacterium of the genus Anaerotignum is increased compared with that in a non-cancer control subject, the test subject is found likely to have kidney cancer of stage I to II, and
when the abundance ratio of the bacterium of the genus Eggerthella is increased compared with that in a non-cancer control subject, the test subject is found likely to have kidney cancer of stage I to II or stage III to IV.
[4] The method for determining kidney cancer according to [3], wherein
the bacterium of the genus Mediterraneibacter includes a bacterium of the genus Mediterraneibacter having 16S rRNA gene having at least 97% sequence identity to the nucleotide sequence set forth in SEQ ID NO: 5,
the bacterium of the genus Ruthenibacterium i Ruthenibacterium lactatiformans having 16S rRNA gene having at least 97% sequence identity to the nucleotide sequence set forth in SEQ ID NO: 6, and a bacterium of the genus Ruthenibacterium having 16S rRNA gene having at least 97% sequence identity to the nucleotide sequence set forth in SEQ ID NO: 7,
the bacterium of the genus Enterocloster includes a bacterium of the genus Enterocloster having 16S rRNA gene having at least 97% sequence identity to the nucleotide sequence set forth in SEQ ID NO: 3, and Clostridium bolteae having 16S rRNA gene having at least 97% sequence identity to the nucleotide sequence set forth in SEQ ID NO: 1,
the bacterium of the genus Anaerotignum includes Anaerotignum lactatifermentans having 16S rRNA gene having at least 97% sequence identity to the nucleotide sequence set forth in SEQ ID NO: 8, and
the bacterium of the genus Eggerthella includes Eggerthella lenta having 16S rRNA gene having at least 97% sequence identity to the nucleotide sequence set forth in SEQ ID NO: 9.
[5] The method for determining kidney cancer according to any one of [1] to [4], wherein the sample is feces.
[6] The method for determining kidney cancer according to any one of [1] to [5], wherein the nucleotide sequence information on the intestinal bacterium-derived genomic DNA is nucleotide sequence information on the 16S rRNA gene.
[7] The method for determining kidney cancer according to any one of [1] to [6], wherein the test subject is a Japanese person.

Other examples of the mode for carrying out the present invention can include:
a method for creating data for determining kidney cancer, comprising the steps (a) and (b-1);
a method for creating data for determining kidney cancer, comprising the steps (a) and (b-2);
a method for diagnosing kidney cancer, comprising the steps (a) and (b-1);
a method for diagnosing kidney cancer, comprising the steps (a) and (b-2);
a method for treating kidney cancer or a method for preventing exacerbation of kidney cancer, comprising the steps (a) and (b-1) and comprising the step of performing a procedure for treating kidney cancer or a procedure for preventing exacerbation of kidney cancer (e.g., drug therapy using an anticancer agent or the like, radiation therapy, surgical treatment, and immunotherapy) on the test subject found likely to have kidney cancer of stage I to II or stage III to IV in the step (b-1); and
a method for treating kidney cancer or a method for preventing exacerbation of kidney cancer, comprising the steps (a) and (b-2) and comprising the step of performing a procedure for treating kidney cancer or a procedure for preventing exacerbation of kidney cancer (e.g., drug therapy using an anticancer agent or the like, radiation therapy, surgical treatment, and immunotherapy) on the test subject found likely to have kidney cancer of stage I to II or stage III to IV in the step (b-2).

### Effect of the Invention

According to the present invention, not only kidney cancer of stage I to II (early kidney cancer) but kidney cancer of stage III to IV (advanced kidney cancer) can be accurately determined. Therefore, the present invention contributes to treatment of kidney cancer and prevention of stage progression of kidney cancer by performing proper treatment on more kidney cancer patients or by performing a proper procedure for preventing exacerbation of kidney cancer.

### BRIEF DESCRIPTION OF THE DRAWINGS

For a more complete understanding of the features and advantages of the present invention, reference is now made to the detailed description of the invention along with the accompanying figures and in which:

FIGS. 1A-E are diagrams showing results of analyzing an abundance ratio of bacteria of five types of genera (Ruminococcus_g4 [FIG. 1A], the genus Ruthenibacterium [FIG. 1B], Clostridium g24 [FIG. 1C], the genus Anaerotignum [FIG. 1D], and the genus Eggerthella [FIG. 1E]) in two types of groups (a control group and a kidney cancer group), relative to all intestinal bacteria. In the drawing, “*” and “**” represent that there is a statistically significant difference (p<0.05 and p<0.01, respectively).

FIGS. 2A-E are diagrams showing results of analyzing an abundance ratio of bacteria of five types of genera (Ruminococcus_g4 [FIG. 2A], the genus Ruthenibacterium [FIG. 2B], Clostridium_g24 [FIG. 2C], the genus Anaerotignum [FIG. 2D], and the genus Eggerthella [FIG. 2E]) in three types of groups (a control group, a localized group, and an advanced group), relative to all intestinal bacteria. In the drawing, “*” represents that there is a statistically significant difference (p<0.05).

FIGS. 3A-D are diagrams showing results of analyzing an abundance ratio of four types of bacteria (Clostridium bolteae [FIG. 3A], PAC001050_s [FIG. 3B], FCEY_s [FIG. 3C], and Anaerotignum lactafifermentas [FIG. 3D]) in two types of groups (a control group and a kidney cancer group), relative to all intestinal bacteria. In the drawing, “*” represents that there is a statistically significant difference (p<0.05).

FIGS. 4A-D are diagrams showing results of analyzing an abundance ratio of four types of bacteria (Clostridium bolteae [FIG. 4A], PAC001050_s [FIG. 4B], FCEY_s [FIG. 4C], and Anaerotignum lactafifermentas [FIG. 4D]) in three types of groups (a control group, a localized group, and an advanced group), relative to all intestinal bacteria. In the drawing, “*” represents that there is a statistically significant difference (p<0.05).

FIG. 5A is a diagram showing results of analyzing an abundance ratio of a bacterium of the genus Ruthenibacterium in two types of groups (a control group and a kidney cancer group), relative to all intestinal bacteria (CLR abundance). FIG. 5B is a diagram showing results of analyzing an abundance ratio of a bacterium of the genus Ruthenibacterium in three types of groups (a control group, a localized group, and an advanced group), relative to all intestinal bacteria (CLR abundance). In the drawing, “*”, “**”, and “***” represent that there is a statistically significant difference (p<0.05, p<0.01, and p<0.001, respectively).

FIGS. 6A-D are diagrams showing results of analyzing the correlation between four types of metabolites (aspartic acid [FIG. 6A], terephthalic acid [FIG. 6B], N-acetylglucosamine [FIG. 6C], and N-acetylneuraminic acid [FIG. 6D]) and an abundance ratio of a bacterium of the genus Ruthenibacterium to all intestinal bacteria (CLR abundance) for three types of groups (a control group [“∘” in the drawing], a localized group [“Δ” in the drawing], and an advanced group [“x” in the drawing]).

FIGS. 7A-D are diagrams showing results of analyzing the correlation between four types of metabolites (N1-acetylspermidine [FIG. 7A], ADMA [FIG. 7B], dodecanoic acid [FIG. 7C], and decanoic acid [FIG. 7D]) and an abundance ratio of a bacterium of the genus Ruthenibacterium to all intestinal bacteria (CLR abundance) for three types of groups (a control group [“∘” in the drawing], a localized group [“Δ” in the drawing], and an advanced group [“x” in the drawing]).

### DETAILED DESCRIPTION OF THE INVENTION

### Mode of Carrying Out the Invention

One embodiment of the method of the present invention is a method for determining kidney cancer, comprising, in order, the steps of:
(a) separating intestinal bacterium-derived genomic DNA from an intestinal bacterium-containing sample collected from a test subject (in the present specification, also referred to as a “test subject-derived sample”); and
(b-1) on the basis of nucleotide sequence information on the intestinal bacterium-derived genomic DNA,
calculating, relative to all intestinal bacteria, an abundance ratio of one or more bacteria (in the present specification, also referred to as the “intestinal bacterium 1 of the present invention”) selected from the group consisting of i) a bacterium of the genus Clostridium having 16S rRNA gene having at least 90% sequence identity to the nucleotide sequence set forth in SEQ ID NO: 1, ii) a bacterium of the genus Eubacterium having 16S rRNA gene having at least 90% sequence identity to the nucleotide sequence set forth in SEQ ID NO: 2, iii) a bacterium of the genus Enterocloster having 16S rRNA gene having at least 95% sequence identity to the nucleotide sequence set forth in SEQ ID NO: 3, and iv) a bacterium of the genus Anaerotignum having 16S rRNA gene having at least 90% sequence identity to the nucleotide sequence set forth in SEQ ID NO: 4, wherein
when the abundance ratio of the bacterium i) is increased compared with that in a non-cancer control subject, the test subject is found likely to have kidney cancer of stage I to II or stage III to IV,
when the abundance ratio of the bacterium ii) is increased compared with that in a non-cancer control subject, the test subject is found likely to have kidney cancer of stage III to IV,
when the abundance ratio of the bacterium iii) is increased compared with that in a non-cancer control subject, the test subject is found likely to have kidney cancer of stage I to II or stage III to IV, and
when the abundance ratio of the bacterium iv) is increased compared with that in a non-cancer control subject, the test subject is found likely to have kidney cancer of stage I to II.

Another embodiment of the method of the present invention is a method for determining kidney cancer, comprising, in order, the steps of:
(a) separating intestinal bacterium-derived genomic DNA from a test subject-derived sample; and
(b-2) on the basis of nucleotide sequence information on the intestinal bacterium-derived genomic DNA,
calculating, relative to all intestinal bacteria, an abundance ratio of one or more bacteria (in the present specification, also referred to as the “intestinal bacterium 2 of the present invention”) selected from the group consisting of a bacterium of the genus Mediterraneibacter, a bacterium of the genus Ruthenibacterium, a bacterium of the genus Enterocloster, a bacterium of the genus Anaerotignum, and a bacterium of the genus Eggerthella, wherein
when the abundance ratio of the bacterium of the genus Mediterraneibacter is increased compared with that in a non-cancer control subject or a patient with kidney cancer of stage I to II, the test subject is found likely to have kidney cancer of stage III to IV,
when the abundance ratio of the bacterium of the genus Ruthenibacterium is increased compared with that in a non-cancer control subject, the test subject is found likely to have kidney cancer of stage III to IV,
when the abundance ratio of the bacterium of the genus Enterocloster is increased compared with that in a non-cancer control subject, the test subject is found likely to have kidney cancer of stage I to II,
when the abundance ratio of the bacterium of the genus Anaerotignum is increased compared with that in a non-cancer control subject, the test subject is found likely to have kidney cancer of stage I to II, and
when the abundance ratio of the bacterium of the genus Eggerthella is increased compared with that in a non-cancer control subject, the test subject is found likely to have kidney cancer of stage I to II or stage III to IV.

The method of the present invention is a method for assisting in diagnosis of kidney cancer by a physician and excludes diagnostic action by a physician. In the present specification, the intestinal bacterium 1 of the present invention and the intestinal bacterium 2 of the present invention are also collectively referred to as the “intestinal bacterium of the present invention”.

As used herein, the “kidney cancer” can be a cancerous condition of cells in the kidney. Examples thereof can include kidney cancer of stage I (i.e., a condition in which the cancer has a diameter of 7 cm or smaller, but is confined within the kidney), kidney cancer of stage II (i.e., a condition in which the cancer has a diameter exceeding 7 cm, but is confined within the kidney), kidney cancer of stage III (i.e., a condition in which the cancer has spread into the vein and/or the perinephrium without extending to the adrenal gland, without invading beyond Gerota's fascia, and without metastasizing to other organs or regional lymph node, or a condition in which the cancer has metastasized to regional lymph node without metastasizing to other organs), and kidney cancer of stage IV (i.e., a condition in which the cancer has spread beyond Gerota's fascia, or a condition in which the cancer has metastasized to other organs) and can preferably include kidney cancer of stage I to II (early kidney cancer) and kidney cancer of stage III to IV (advanced kidney cancer).

As used herein, the “intestinal bacterium-containing sample collected from a test subject” can be a test subject-derived biological sample containing an intestinal bacterium. Examples thereof can include feces and an intestinal mucosal tissue and can preferably include feces because of easy collection and low invasiveness.

As used herein, the “test subject” can be a human and is preferably a human of the culture in which rice is a staple (e.g., people in East Asia, Central Asia, South Asia, Southeast Asia, and West Asia), more preferably Mongoloid (e.g., Southern Mongoloid, Northern neo-Mongoloid, Central neo-Mongoloid, Southern neo-Mongoloid, Inuit, and Amerind). Examples thereof can specifically include a Japanese person, a Korean person, a Chinese person, a Taiwanese person, a Mongolian person, Tibetans, Indochinese people, and a Native American and can preferably include a Japanese person.

As used herein, the “non-cancer control subject” can be a human having no cancer and is preferably a human of the same race as that of the test subject to be compared.

As used herein, the “at least 90% sequence identity to the nucleotide sequence set forth in SEQ ID NO: X” means that a sequence is identical to 90% or higher of the whole nucleotide sequence of SEQ ID NO: X by the substitution, deletion, insertion, addition, or inversion (hereinafter, also referred to as “substitution or the like”) of one or several nucleotides in the nucleotide sequence of SEQ ID NO: X, or a sequence is 100% identical to the whole nucleotide sequence of SEQ ID NO: X without such substitution or the like. As used herein, the “at least 95% sequence identity to the nucleotide sequence set forth in SEQ ID NO: 3” means that a sequence is identical to 95% or higher of the whole nucleotide sequence of SEQ ID NO: 3 by the substitution or the like of one or several nucleotides in the nucleotide sequence of SEQ ID NO: 3, or a sequence is 100% identical to the whole nucleotide sequence of SEQ ID NO: 3 without such substitution or the like. In this context, the “nucleotide sequence derived by the substitution, deletion, insertion, addition, or inversion of one or several nucleotides” means a nucleotide sequence derived by the substitution, deletion, insertion, addition, or inversion of the number of nucleotides, for example, within the range of 1 to 149, preferably within the range of 1 to 100, more preferably within the range of 1 to 75, still more preferably within the range of 1 to 50, even more preferably within the range of 1 to 40, further preferably within the range of 1 to 30, still further preferably within the range of 1 to 15.

As used herein, the “at least 90% sequence identity” means that the sequence identity is 90% or higher, preferably the sequence identity is 91% or higher, more preferably the sequence identity is 92% or higher, still more preferably the sequence identity is 93% or higher, even more preferably the sequence identity is 94% or higher, particularly preferably the sequence identity is 95% or higher, particularly more preferably the sequence identity is 96% or higher, particularly still more preferably the sequence identity is 97% or higher, particularly even more preferably the sequence identity is 98% or higher, most preferably the sequence identity is 99% or higher (e.g., 99.1% or higher, 99.2% or higher, 99.3% or higher, 99.4% or higher, 99.5% or higher, 99.6% or higher, 99.7% or higher, 99.8% or higher, 99.9% or higher, or 100%). As used herein, the “at least 95% sequence identity” means that the sequence identity is 95% or higher, preferably the sequence identity is 96% or higher, more preferably the sequence identity is 97% or higher, still more preferably the sequence identity is 98% or higher, even more preferably the sequence identity is 99% or higher (e.g., 99.1% or higher, 99.2% or higher, 99.3% or higher, 99.4% or higher, 99.5% or higher, 99.6% or higher, 99.7% or higher, 99.8% or higher, 99.9% or higher, or 100%). The identity of a nucleotide sequence can be determined through the use of BLASTX based on algorithm BLAST by Karlin and Altschul (Proc. Natl. Acad. Sci. USA 87:2264-2268, 1990; and Proc Natl Acad Sci USA 90:5873, 1993) or a program called BLASTN (Altschul S F, et al: J Mol Biol 215:403, 1990) based on a program called BLASTP (Altschul S F, et al: J Mol Biol 215:403, 1990). In the case of analyzing a nucleotide sequence using BLASTN, parameters are set to, for example, score=100 and word length=12.

Examples of the bacterium i) as the intestinal bacterium 1 of the present invention can more specifically include Clostridium bolteae having 16S rRNA gene having at least 97% sequence identity to the nucleotide sequence set forth in SEQ ID NO: 1. Examples of the bacterium ii) can more specifically include Eubacterium coprostanoligenes having 16S rRNA gene having at least 97% sequence identity to the nucleotide sequence set forth in SEQ ID NO: 2. Examples of the bacterium iii) can more specifically include a bacterium of the genus Enterocloster (e.g., FCEY_s) having 16S rRNA gene having at least 97% sequence identity to the nucleotide sequence set forth in SEQ ID NO: 3. Examples of the bacterium iv) can more specifically include Anaerotignum lactafifermentas having 16S rRNA gene having at least 97% sequence identity to the nucleotide sequence set forth in SEQ ID NO: 4.

Examples of the bacterium of the genus Mediterraneibacter as the intestinal bacterium 2 of the present invention can include a bacterial population of the genus Mediterraneibacter including a bacterium of the genus Mediterraneibacter having 16S rRNA gene having at least 90% sequence identity to the nucleotide sequence set forth in SEQ ID NO: 5, and Mediterraneibacter massiliensis, and more specifically include a bacterium of the genus Mediterraneibacter (LT635549_s) having 16S rRNA gene having at least 97% sequence identity to the nucleotide sequence of SEQ ID NO: 5. Examples of the bacterium of the genus Ruthenibacterium as the intestinal bacterium 2 of the present invention can include a bacterial population of the genus Ruthenibacterium including a bacterium of the genus Ruthenibacterium (e.g., Ruthenibacterium lactatiformans) having 16S rRNA gene having at least 90% sequence identity to the nucleotide sequence set forth in SEQ ID NO: 6, and a bacterium of the genus Ruthenibacterium having 16S rRNA gene having at least 90% sequence identity to the nucleotide sequence set forth in SEQ ID NO: 7, and more specifically include Ruthenibacterium lactatiformans having 16S rRNA gene having at least 97% sequence identity to the nucleotide sequence of SEQ ID NO: 6, and a bacterium of the genus Ruthenibacterium (PAC001646_s) having 16S rRNA gene having at least 97% sequence identity to the nucleotide sequence of SEQ ID NO: 7. Examples of the bacterium of the genus Enterocloster as the intestinal bacterium 2 of the present invention can include a bacterial population of the genus Enterocloster including a bacterium of the genus Enterocloster having 16S rRNA gene having at least 90% sequence identity to the nucleotide sequence set forth in SEQ ID NO: 3, and a bacterium (e.g., Clostridium bolteae) having 16S rRNA gene having at least 90% sequence identity to the nucleotide sequence set forth in SEQ ID NO: 1, and more specifically include a bacterium of the genus Enterocloster having 16S rRNA gene having at least 97% sequence identity to the nucleotide sequence of SEQ ID NO: 3, and Clostridium bolteae (document “Int J Syst Evol Microbiol. 2020 January; 70 (1): 23-34”) having 16S rRNA gene having at least 97% sequence identity to the nucleotide sequence of SEQ ID NO: 1. Examples of the bacterium of the genus Anaerotignum as the intestinal bacterium 2 of the present invention can include a bacterial population of the genus Anaerotignum including a bacterium of the genus Anaerotignum having 16S rRNA gene having at least 90% sequence identity to the nucleotide sequence set forth in SEQ ID NO: 8, and Anaerotignum aminivorans, and more specifically include Anaerotignum lactatifermentans having 16S rRNA gene having at least 97% sequence identity to the nucleotide sequence of SEQ ID NO: 8. Examples of the bacterium of the genus Eggerthella as the intestinal bacterium 2 of the present invention can include a bacterial population of the genus Eggerthella including a bacterium of the genus Eggerthella having 16S rRNA gene having at least 90% sequence identity to the nucleotide sequence set forth in SEQ ID NO: 9, and Eggerthella lenta, and more specifically include Eggerthella lenta having 16S rRNA gene having at least 97% sequence identity to the nucleotide sequence of SEQ ID NO: 9.

In the step (a), a method for separating the intestinal bacterium-derived genomic DNA from the test subject-derived sample is not particularly limited. For example, the test subject-derived sample is first suspended in a buffer solution (e.g., Tris and HEPES) containing a digestive enzyme (e.g., a proteolytic enzyme such as protease K, and a polysaccharide degrading enzyme), a denaturant (e.g., guanidine hydrochloride and urea), a surfactant (e.g., SDS and Triton X-100), a chelating agent (e.g., EDTA and EGTA), and the like, and bacteriolytic treatment of the intestinal bacterium is performed. In order to effectively perform the bacteriolytic treatment, beads for physically disrupting cells may be added to the suspension, and disruption treatment with the beads can be performed. Examples of the material of the beads used can include, but are not particularly limited to, glass, guanidium, garnet, zirconium, silica, and a combination thereof.

Next, the intestinal bacterium-derived genomic DNA is separated from the intestinal bacterium lysate after the bacteriolytic treatment. Examples of such a separation method can include a method known in the art, for example, a method of denaturing and removing a component other than a nucleic acid (e.g., a phenol/chloroform method and a sodium iodide method), a method of allowing a nucleic acid to be adsorbed to a silica membrane, a magnetic glass particle, or the like (e.g., a spin column method), and a method based on a combination thereof. Alternatively, the intestinal bacterium-derived genomic DNA may be separated from the test subject-derived sample using a commercially available kit (e.g., Nucleospin microbial DNA kit [manufactured by MACHEREY-NAGEL GmbH & Co. KG], Stool DNA Isolation Kit [manufactured by Norgen Biotek Corp.], or NucleoSpin DNA Stool [manufactured by Takara Bio Inc.]).

In the steps (b-1) and (b-2), examples of the method for calculating the abundance ratio of the intestinal bacterium of the present invention to all intestinal bacteria on the basis of nucleotide sequence information on the intestinal bacterium-derived genomic DNA can include a method of performing PCR using a primer set (which consists of a forward primer and a reverse primer) designed so as to amplify a region in the genomic DNA specific for each intestinal bacterium, subjecting the obtained PCR product to sequence analysis (e.g., multiplex analysis, single-read analysis, and a paired-end analysis) using a sequencer such as a next generation sequencer, and measuring a ratio of the genomic DNA of the intestinal bacterium of the present invention to the genomic DNA derived from all the intestinal bacteria on the basis of the obtained nucleotide sequence information.

A method for identifying the intestinal bacterium of the present invention from the nucleotide sequence information after the sequence analysis is not particularly limited, and an analysis method known in the art using analytical software, a database, or the like can be utilized. In the case of using a database, the intestinal bacterium of the present invention can be identified by, for example, sequence identity search for the nucleotide sequence of the intestinal bacterium of the present invention maintained in the database. Specific examples of the database can include GenBank, ENA, and DDBJ. Alternatively, QIIME (Quantitative Insights in Microbial Ecology) may be used, which is open source software of intestinal flora analysis.

Examples of the “region in the genomic DNA specific for each intestinal bacterium” can include a gene encoding 5S rRNA (5S rRNA gene [also referred to as 5S rDNA]), a gene encoding 16S rRNA (16S rRNA gene [also referred to as 16S rDNA]), and a gene encoding 23S rRNA (23S rRNA gene [also referred to as 23S rDNA]) and can preferably include 16S rRNA gene because effects thereof have been demonstrated in the present Examples mentioned later.

Specifically, examples of the “nucleotide sequence information on the intestinal bacterium-derived genomic DNA” can include nucleotide sequence information on the 5S rRNA gene, nucleotide sequence information on the 16S rRNA gene, and nucleotide sequence information on the 23S rRNA gene and can preferably include nucleotide sequence information on the 16S rRNA gene because effects thereof have been demonstrated in the present Examples mentioned later.

The “16S rRNA gene” may be the whole 16S rRNA gene or may be a portion thereof (e.g., a region differing in nucleotide sequence among types of bacteria [specifically, one or more regions selected from a V1 region, a V2 region, a V3 region, a V4 region, a V5 region, a V6 region, a V7 region, a V8 region, and a V9 region]). Examples thereof can preferably include a V1 region and a V2 region of the 16S rRNA gene because effects thereof have been demonstrated in the present Examples mentioned later. In this context, in the case of amplifying the V1 region and the V2 region by PCR, primer sets may be designed so as to hybridize to sites flanking the V1 region and the V2 region, respectively. Alternatively, since both the regions are linked to each other, a primer set can be designed so as to hybridize to sites flanking the V1 region located on the upstream side and the V2 region located on the downstream side. This approach is efficient and preferable because only one primer set is used and also because PCR is performed only once.

As used herein, the “upstream side” means the start codon (ATG) side of the 16S rRNA gene, and the “downstream side” means the stop codon (TGA, TAG, or TAA) side of the 16S rRNA gene.

In the step (b-1), when the abundance ratio of the bacterium iv) is decreased compared with that in a patient with kidney cancer of stage I to II, the test subject may be regarded as being likely to have kidney cancer of stage III to IV by using this indicator. In the step (b-2), when the abundance ratio of the bacterium of the genus Enterocloster is decreased compared with that in a patient with kidney cancer of stage I to II, the test subject may be regarded as being likely to have kidney cancer of stage III to IV by using this indicator.

Hereinafter, the present invention will be more specifically described with reference to Examples. However, the technical scope of the present invention is not limited by these examples.

### Example 1

### 1. Method

### 1-1 Kidney Cancer Group and Control Group

A kidney cancer group involved 38 kidney cancer cases surgically operated in initial treatment and hospitalized for the purpose of systemic treatment in the Department of Urology at the Tohoku University for a period from December 2020 to February 2021 (Table 1). These patients had no recent history of antibiotic administration. A control group involved 51 cases of patients having eGFR of 30 mL/min/1.73 m² or more without diabetes mellitus as a complication among patients visiting the Department of Nephrology and Hypertension at the Tohoku University as outpatients (Table 1). All the 38 kidney cancer cases were pathologically diagnosed with kidney cancer, 35 cases of which had ccRCC, and 3 cases of which had papillary renal cell carcinoma (pRCC) (Table 1). Kidney cancer patients diagnosed with stage III or higher by TNM classification were classified into an advanced group, and kidney cancer patients diagnosed with stage I to II were classified into a localized group. All the patients in the control group and the kidney cancer group were Japanese.

TABLE 1

| | | Control | Kidney |
|---|---|---|---|
| | | group | cancer group |
| | | N = 51 | N = 38 |
| Sex | Male | 31(61%) | 25(66%) |
| | Female | 20(39%) | 13(34%) |
| Age | Average | 72 | 66.5 |
| | value | | |
| | Range | 36-94 | 48-89 |
| BMI | Average | 24.0 | 23.1 |
| (kg/m²) | value | | |
| | Range | 17.3-37.8 | 17.6-32.1 |
| Hypertension | | 45(88%) | 20(53%) |
| (percentage) | | | |
| Diabetes mellitus | | 0 | 4(10%) |
| (percentage) | | | |
| eGFR | Average | 64 | 56 |
| (mL/min/1.73 m²) | value | | |
| | Range | 30-117 | 30-83 |
| TNM Classification | Stage I | — | 17 |
| | Stage II | — | 2 |
| | Stage III | — | 7 |
| | Stage IV | — | 12 |
| Metastasis | | — | 13(34%) |
| (percentage) | | | |
| Pathological | ccRCC | — | 35(92%) |
| classification | pRCC | — | 3(8%) |
| (percentage) | | | |

### 1-2 Separation of Intestinal Bacterium-Derived Genomic DNA

Feces were collected from each of the kidney cancer group and the control group before surgery or treatment, and intestinal bacterium-derived genomic DNA was separated using Nucleospin microbial DNA kit (manufactured by MACHEREY-NAGEL GmbH & Co. KG). Specifically, 100 μL of PBS containing 0.2 to 0.5 g of the feces and 500 μL of an elution buffer included in the kit were mixed and added, together with protease K, to Nucleospin beads tube (manufactured by MACHEREY-NAGEL GmbH & Co. KG) containing ceramic beads so that cells were disrupted with the beads. Subsequent operation was carried out in accordance with the instruction manual of the kit to separate intestinal bacterium-derived genomic DNA. 1-3 Sequence analysis of 16S rRNA gene in intestinal bacterium-derived genomic DNA

In order to determine the sequence of the 16S rRNA gene of the separated intestinal bacterium-derived genomic DNA, analysis was conducted using a next generation sequencer (MiSeq, manufactured by Illumina, Inc.). Specifically, PCR was performed with the intestinal bacterium-derived genomic DNA (1 ng) as a template using a primer set for the first PCR (1st_PCR_27Fmod MIX and 1st_PCR_338R_MIX [both manufactured by Illumina, Inc.]) and EX Taq HS (manufactured by Takara Bio Inc.) in accordance with the protocols attached to the products to perform the amplification of V1 to V2 regions of the 16S rRNA gene and the addition of an adapter sequence. Next, PCR was performed with the obtained PCR product (approximately 430 bases long) as a template using a primer set for the second PCR (2nd Forward primer and 2nd Reverse primer [both manufactured by Illumina, Inc.]) and EX Taq HS (manufactured by Takara Bio Inc.) in accordance with the protocols attached to the products to perform the amplification of the V1 to V2 regions of the 16S rRNA gene and the addition of an adapter sequence for sequencing and an index for sample identification. Then, sequence analysis was conducted by the paired-end method using a next generation sequencer (MiSeq, manufactured by Illumina, Inc.).

1-4 Identification of Genus and Species of Bacterium from which 16S rRNA Gene is Derived

On the basis of the obtained sequence analysis data, OTU (operational taxonomic unit) was created, and homology to a representative sequence was evaluated using QIIME (Quantitative Insights in Microbial Ecology), which is open source software of intestinal flora analysis, to obtain bacterial systematic classification data. For intestinal bacterium classification, an expression frequency was analyzed on class, order, family, genus, and species bases, starting from the upper rank phylum. Each group was tested such that a difference in relative abundance of OTU was 1% or more. A sample having a relative abundance of less than 1% was excluded. In analysis at the genus level and the species level, an LDA effect size was calculated using LEfSe (linear discriminant analysis effect size) to search for a significantly different biomarker (intestinal bacterium). The Wilcoxon/Kruskal-Wallis test was used for the comparison between two groups (the control group and the kidney cancer group), and the Turkey-Kramer test was used for the comparison among three groups (the control group, the localized group, and the advanced group). An intestinal bacterium having the 16S rRNA gene (V1 to V2 regions) having 97% or higher sequence identity to the nucleotide sequence of the 16S rRNA gene (V1 to V2 regions) of a bacterium of known species was classified into an intestinal bacterium of the same species as the known species. ALDEx2 (ANOVA-Like Differential Expression analysis version 2) analysis was further added to identify a bacterial species increased in the kidney cancer group.

### 2. Results

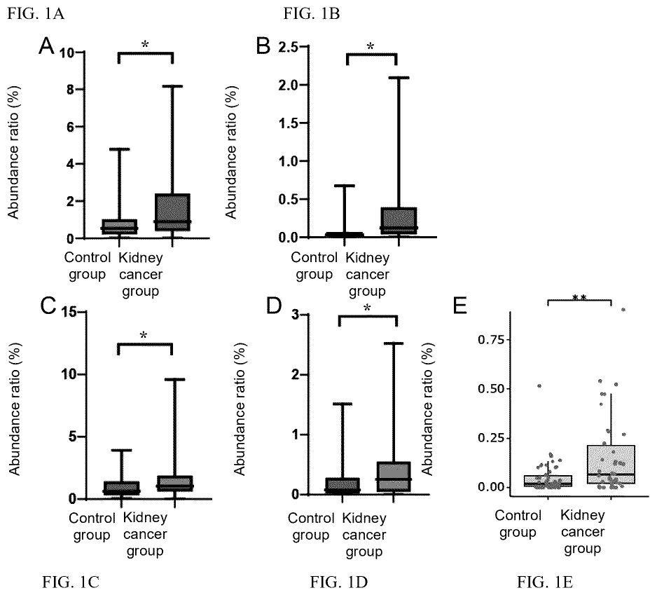

As a result, bacteria of five types of genera (Ruminococcus g4, the genus Ruthenibacterium, Clostridium_g24, the genus Anaerotignum, and the genus Eggerthella) were identified, by analysis at the genus level, as intestinal bacteria, the abundance ratio of which relative to all intestinal bacteria in the kidney cancer group was confirmed to be significantly increased in comparison with the abundance ratio in the control group (FIG. 1). Among them, the Ruminococcus g4 bacterium is a bacterium of the genus Mediterraneibacter including a bacterium (LT635549_s) having the 16S rRNA gene having the nucleotide sequence of SEQ ID NO: 5, and the like. The bacterium of the genus Ruthenibacterium is a bacterium of the genus Ruthenibacterium including Ruthenibacterium lactatiformans having the 16S rRNA gene having the nucleotide sequence of SEQ ID NO: 6, PAC001646_s having the 16S rRNA gene having the nucleotide sequence of SEQ ID NO: 7, and the like. The Clostridium_g24 bacterium is a bacterium of the genus Enterocloster including FCEY_s mentioned later, Clostridium bolteae mentioned later, and the like. The bacterium of the genus Anaerotignum is a bacterium of the genus Anaerotignum including Anaerotignum lactatifermentans having the 16S rRNA gene having the nucleotide sequence of SEQ ID NO: 8, and the like. The bacterium of the genus Eggerthella is a bacterium of the genus Eggerthella including Eggerthella lenta having the 16S rRNA gene having the nucleotide sequence of SEQ ID NO: 9, and the like.

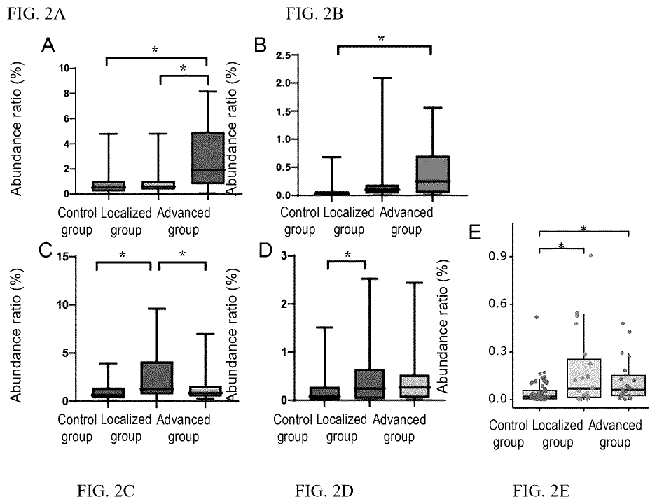

As a result of comparing the bacteria of five types of genera among three groups (the control group, the localized group, and the advanced group), the abundance ratio of the Ruminococcus_g4 bacterium exhibited significant increase in the advanced group compared with the control group or the localized group (FIG. 2A). The abundance ratio of the bacterium of the genus Ruthenibacterium exhibited significant increase in the advanced group compared with the control group (FIG. 2B). The abundance ratio of the Clostridium g24 bacterium exhibited significant increase in the localized group compared with the control group and significant decrease in the advanced group compared with the localized group (FIG. 2C). The abundance ratio of the bacterium of the genus Anaerotignum exhibited significant increase in the localized group compared with the control group (FIG. 2D). The abundance ratio of the bacterium of the genus Eggerthella exhibited significant increase in the localized group or the advanced group compared with the control group (FIG. 2E).

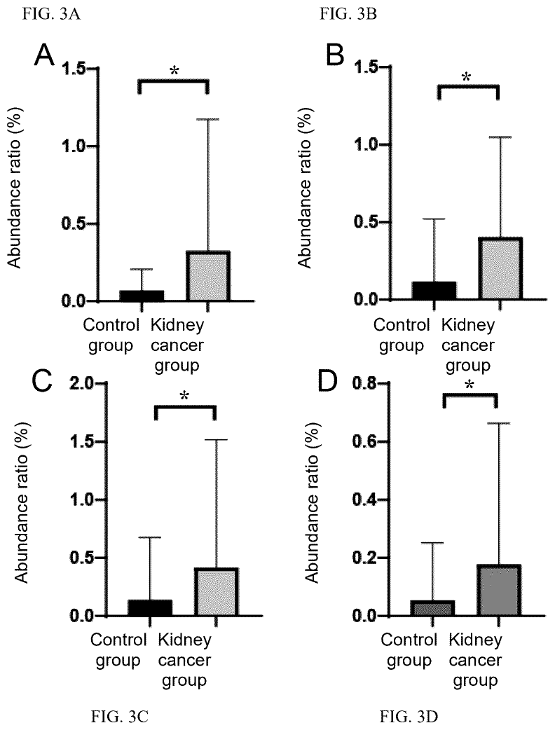

Four types of bacteria (Clostridium bolteae, PAC001050_s, FCEY_s, and Anaerotignum lactafifermentas) were identified, by analysis at the species level, as intestinal bacteria, the abundance ratio of which relative to all intestinal bacteria in the kidney cancer group was confirmed to be significantly increased in comparison with the abundance ratio in the control group (FIG. 3). Among them, Clostridium bolteae is a bacterium having the 16S rRNA gene having the nucleotide sequence of SEQ ID NO: 1. PAC001050_s described above is Eubacterium coprostanoligenes having the 16S rRNA gene having the nucleotide sequence of SEQ ID NO: 2. FCEY_s described above is a bacterium of the genus Enterocloster having the 16S rRNA gene having the nucleotide sequence of SEQ ID NO: 3, and the nucleotide sequence of its genomic DNA has been registered (NCBI RefSeq assembly; GCF_001517625.2). FCEY_s described above, which was formerly regarded as Clostridium bouchedurhonense, has recently been classified into the same bacterium of the genus Enterocloster as that of Enterocloster alcoholdehydrogenati according to the classification of GTDB (The Genome Taxonomy Database) (URL “https://gtdb.ecogenomic.org/genome?gid=GCA_001517625.2”). Thus, FCEY_s described above is a bacterium of the genus Enterocloster including Enterocloster alcoholdehydrogenati or might be a bacterium of a novel genus different from the existing genus Enterocloster. Anaerotignum lactafifermentas described above is a bacterium having the 16S rRNA gene having the nucleotide sequence of SEQ ID NO: 4.

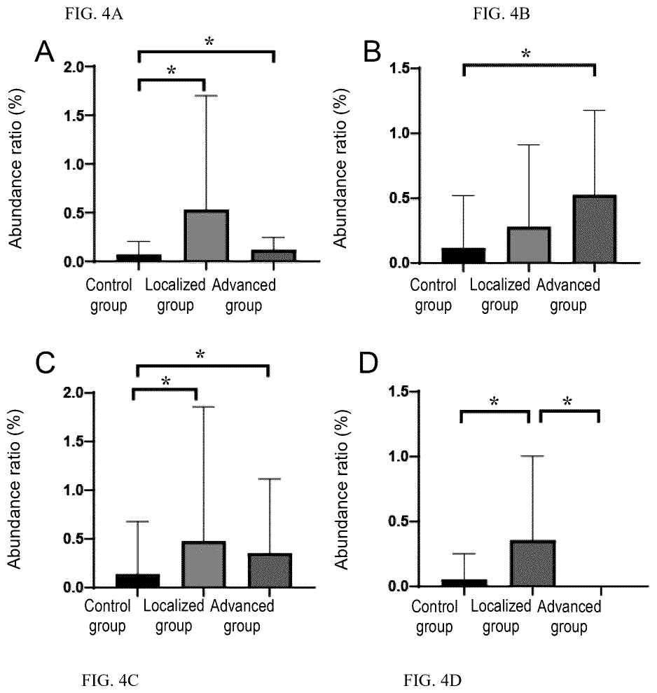

As a result of comparing the four types of bacteria among three groups (the control group, the localized group, and the advanced group), the abundance ratio of Clostridium bolteae exhibited significant increase in the localized group or the advanced group compared with the control group (FIG. 4A). The abundance ratio of PAC001050_s exhibited significant increase in the advanced group compared with the control group (FIG. 4B). The abundance ratio of FCEY_s exhibited significant increase in the localized group or the advanced group compared with the control group (FIG. 4C). The abundance ratio of Anaerotignum lactafifermentas exhibited significant increase in the localized group compared with the control group and significant decrease in the advanced group compared with the localized group (FIG. 4D).

### Example 2

### 1. Identification of Genus of Bacterium Using Approach Different from Classification Analysis in Example 1

In order to analyze the abundance ratio of the bacterium of the genus Ruthenibacterium to all intestinal bacteria, SILVA database classification analysis using ASV (amplicon sequence variant) was conducted instead of the OTU classification analysis using QIIME in the section “1-4” of Example 1. ASV was created using DADA2 (version 1.36.0), and the calculation of an a diversity index, the calculation of a β diversity index, the calculation of the abundance ratio converted to CLR (centered log-ratio) (CLR abundance), and multivariate analysis were performed by standard approaches. In Spearman's rank correlation coefficients of Example 2, “*” “**”, “***”, and “****” represent that there is a statistically significant difference (p<0.05, p<0.01, p<0.001, and p<0.0001, respectively).

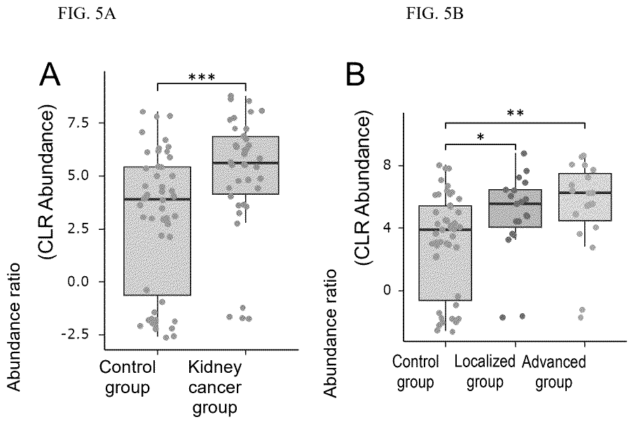

As a result, the abundance ratio of the bacterium of the genus Ruthenibacterium to all intestinal bacteria in the kidney cancer group was increased with a more marked significant difference in comparison with the abundance ratio in the control group (FIG. 5A). The abundance ratio of the bacterium of the genus Ruthenibacterium to all intestinal bacteria in the advanced group was increased with a more marked significant difference in comparison with the abundance ratio in the control group (FIG. 5B). In addition, the abundance ratio of the bacterium of the genus Ruthenibacterium to all intestinal bacteria exhibited a significant positive correlation with the stage progression of kidney cancer (p=0.39***).

### 2. Untargeted Metabolomics

Plasma samples and urine samples were collected from the kidney cancer group and the control group (Table 1), and untargeted metabolomics was conducted using an ultrahigh-performance liquid chromatograph-quadrupole time-of-flight mass spectrometer (UHPLC-QTOF/MS; Waters Synapt G2-Si). Each sample was treated for the removal of proteins and analyzed both in a cationization mode and in an anionization mode. A metabolite was annotated by matching an accurate mass and an MS/MS spectrum to published databases (HMDB [Human Metabolome Database], ChemSpider, and LipidMaps). In order to identify a group-specific metabolic signature, multivariate analysis including PCA (principal component analysis) and OPLS-DA (orthogonal partial least squares discriminant analysis) was conducted.

As a result, six types of metabolites (aspartic acid, terephthalic acid, N-acetylglucosamine, N1-acetylspermidine, and ADMA [asymmetric dimethylarginine] [all derived from the plasma samples], and N-acetylneuraminic acid [derived from the urine samples]) were identified as metabolites confirmed to be significantly increased in the plasma samples or the urine samples in the kidney cancer group in comparison with the samples in the control group and to be significantly increased in the advanced group compared with the localized group. Two types of metabolites (dodecanoic acid and decanoic acid) were identified as metabolites confirmed to be significantly decreased in the plasma samples in the kidney cancer group in comparison with the plasma samples in the control group and to be significantly decreased in the advanced group compared with the localized group.

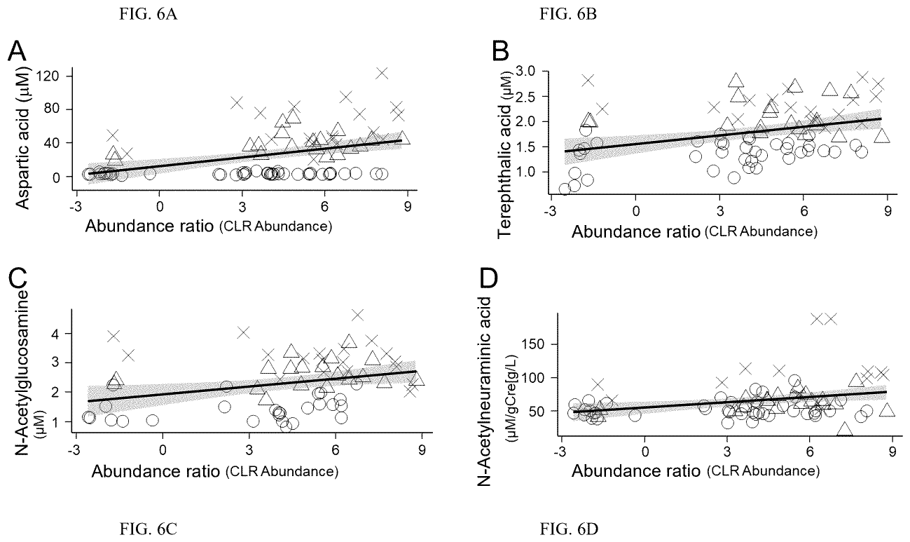

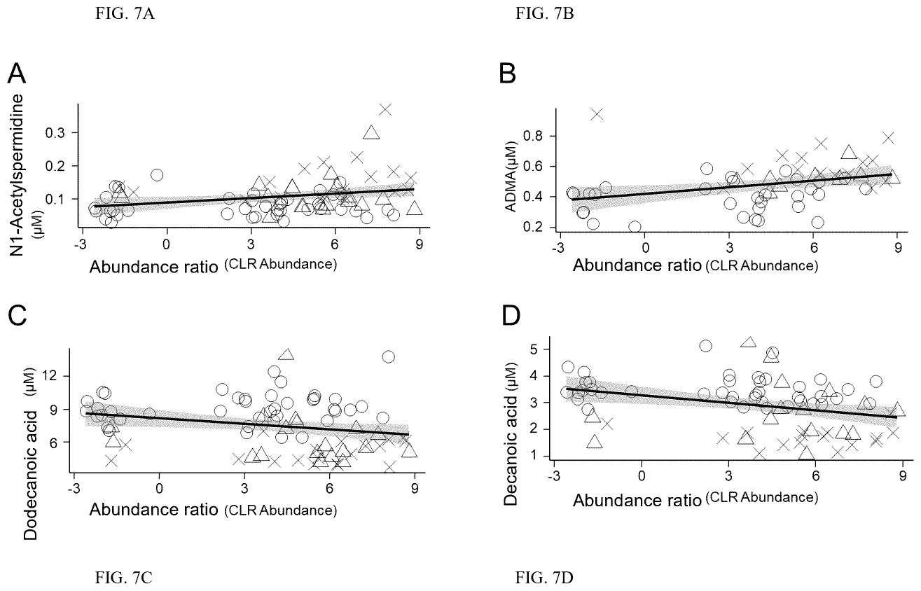

Accordingly, three types of groups (the control group, the localized group, and the advanced group) were analyzed for the correlation between these eight types of metabolites and the abundance ratio of the bacterium of the genus Ruthenibacterium to all intestinal bacteria calculated in FIG. 5, on the basis of Spearman's rank correlation coefficients. As a result, as shown in FIGS. 6 and 7, all of these eight types of metabolites exhibited a significant correlation with the abundance ratio of the bacterium of the genus Ruthenibacterium to all intestinal bacteria (aspartic acid: p=0.44****, terephthalic acid: p=0.35**, N-acetylglucosamine: p=0.38, N-acetylneuraminic acid: p=0.30**, N1-acetylspermidine: p=0.28*, ADMA: p=0.47***, dodecanoic acid: p=0.33**, and decanoic acid: p=0.39***).

These results indicate that the onset of kidney cancer and metabolic change associated with stage progression are related to change in the abundance ratio of the bacterium of the genus Ruthenibacterium to all intestinal bacteria.

### INDUSTRIAL APPLICABILITY

The present invention contributes to early detection of kidney cancer, early treatment of kidney cancer, and prevention of stage progression of kidney cancer.

### SEQUENCE LISTING

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### PRIOR ART DOCUMENTS

### Patent Documents

[Patent Document 1] Japanese unexamined Patent Application Publication No. 2022-76117
[Patent Document 2] Japanese unexamined Patent Application Publication No. 2023-4411

### Non-Patent Document

[Non-patent Document 1] Front Microbiol. 2022 Jul. 4; 13:913718

What is claimed is:

1. A method for treating kidney cancer or a method for preventing exacerbation of kidney cancer, comprising the following steps (a), (b-1), and (c-1):
(a) separating intestinal bacterium-derived genomic DNA from an intestinal bacterium-containing sample collected from a test subject;
(b-1) on the basis of nucleotide sequence information on the intestinal bacterium-derived genomic DNA,
calculating, relative to all intestinal bacteria, an abundance ratio of one or more bacteria selected from the group consisting of i) a bacterium of the genus Clostridium having 16S rRNA gene having at least 90% sequence identity to the nucleotide sequence set forth in SEQ ID NO: 1, ii) a bacterium of the genus Eubacterium having 16S rRNA gene having at least 90% sequence identity to the nucleotide sequence set forth in SEQ ID NO: 2, iii) a bacterium of the genus Enterocloster having 16S rRNA gene having at least 95% sequence identity to the nucleotide sequence set forth in SEQ ID NO: 3, and iv) a bacterium of the genus Anaerotignum having 16S rRNA gene having at least 90% sequence identity to the nucleotide sequence set forth in SEQ ID NO: 4, wherein
when the abundance ratio of the bacterium i) is increased compared with that in a non-cancer control subject, the test subject is diagnosed as being likely to have kidney cancer of stage I to II or stage III to IV,
when the abundance ratio of the bacterium ii) is increased compared with that in a non-cancer control subject, the test subject is found likely to have kidney cancer of stage III to IV, when the abundance ratio of the bacterium iii) is increased compared with that in a non-cancer control subject, the test subject is diagnosed as being likely to have kidney cancer of stage I to II or stage III to IV, and
when the abundance ratio of the bacterium iv) is increased compared with that in a non-cancer control subject, the test subject is diagnosed as being likely to have kidney cancer of stage I to II; and
(c-1) performing a procedure for treating kidney cancer or a procedure for preventing exacerbation of kidney cancer on the test subject diagnosed as being likely to have kidney cancer of stage I to II or stage III to IV in the step (b-1).

2. The method for treating kidney cancer or the method for preventing exacerbation of kidney cancer according to claim 1, wherein
the bacterium i) is Clostridium bolteae having 16S rRNA gene having at least 97% sequence identity to the nucleotide sequence set forth in SEQ ID NO: 1,
the bacterium ii) is Eubacterium coprostanoligenes having 16S rRNA gene having at least 97% sequence identity to the nucleotide sequence set forth in SEQ ID NO: 2,
the bacterium iii) is a bacterium of the genus Enterocloster having 16S rRNA gene having at least 97% sequence identity to the nucleotide sequence set forth in SEQ ID NO: 3, and
the bacterium iv) is Anaerotignum lactafifermentas having 16S rRNA gene having at least 97% sequence identity to the nucleotide sequence set forth in SEQ ID NO: 4.

3. A method for treating kidney cancer or a method for preventing exacerbation of kidney cancer, comprising the following steps (a), (b-2), and (c-2):
(a) separating intestinal bacterium-derived genomic DNA from an intestinal bacterium-containing sample collected from a test subject;
(b-2) on the basis of nucleotide sequence information on the intestinal bacterium-derived genomic DNA,
calculating, relative to all intestinal bacteria, an abundance ratio of one or more bacteria selected from the group consisting of a bacterium of the genus Mediterraneibacter, a bacterium of the genus Ruthenibacterium, a bacterium of the genus Enterocloster, a bacterium of the genus Anaerotignum, and a bacterium of the genus Eggerthella, wherein
when the abundance ratio of the bacterium of the genus Mediterraneibacter is increased compared with that in a non-cancer control subject or a patient with kidney cancer of stage I to II, the test subject is diagnosed as being likely to have kidney cancer of stage III to IV,
when the abundance ratio of the bacterium of the genus Ruthenibacterium is increased compared with that in a non-cancer control subject, the test subject is diagnosed as being likely to have kidney cancer of stage III to IV,
when the abundance ratio of the bacterium of the genus Enterocloster is increased compared with that in a non-cancer control subject, the test subject is found likely to have kidney cancer of stage I to II,
when the abundance ratio of the bacterium of the genus Anaerotignum is increased compared with that in a non-cancer control subject, the test subject is diagnosed as being likely to have kidney cancer of stage I to II, and
when the abundance ratio of the bacterium of the genus Eggerthella is increased compared with that in a non-cancer control subject, the test subject is diagnosed as being likely to have kidney cancer of stage I to II or stage III to IV; and
(c-2) performing a procedure for treating kidney cancer or a procedure for preventing exacerbation of kidney cancer on the test subject diagnosed as being likely to have kidney cancer of stage I to II or stage III to IV in the step (b-2).

4. A method for treating kidney cancer or a method for preventing exacerbation of kidney cancer, comprising the following steps (a), (b-3), and (c-3):
(a) separating intestinal bacterium-derived genomic DNA from an intestinal bacterium-containing sample collected from a test subject;
(b-3) on the basis of nucleotide sequence information on the intestinal bacterium-derived genomic DNA,
calculating, relative to all intestinal bacteria, an abundance ratio of a bacterium of the genus Ruthenibacterium, wherein when the abundance ratio of the bacterium of the genus Ruthenibacterium is increased compared with that in a non-cancer control subject, the test subject is diagnosed as being likely to have kidney cancer of stage III to IV; and
(c-3) performing a procedure for treating kidney cancer or a procedure for preventing exacerbation of kidney cancer on the test subject diagnosed as being likely to have kidney cancer of stage III to IV in the step (b-3).

5. The method for treating kidney cancer or the method for preventing exacerbation of kidney cancer according to claim 3, wherein
the bacterium of the genus Mediterraneibacter includes a bacterium of the genus Mediterraneibacter having 16S rRNA gene having at least 97% sequence identity to the nucleotide sequence set forth in SEQ ID NO: 5,
the bacterium of the genus Ruthenibacterium includes Ruthenibacterium lactatiformans having 16S rRNA gene having at least 97% sequence identity to the nucleotide sequence set forth in SEQ ID NO: 6, and a bacterium of the genus Ruthenibacterium having 16S rRNA gene having at least 97% sequence identity to the nucleotide sequence set forth in SEQ ID NO: 7,
the bacterium of the genus Enterocloster includes a bacterium of the genus Enterocloster having 16S rRNA gene having at least 97% sequence identity to the nucleotide sequence set forth in SEQ ID NO: 3, and Clostridium bolteae having 16S rRNA gene having at least 97% sequence identity to the nucleotide sequence set forth in SEQ ID NO: 1,
the bacterium of the genus Anaerotignum includes Anaerotignum lactatifermentans having 16S rRNA gene having at least 97% sequence identity to the nucleotide sequence set forth in SEQ ID NO: 8, and
the bacterium of the genus Eggerthella includes Eggerthella lenta having 16S rRNA gene having at least 97% sequence identity to the nucleotide sequence set forth in SEQ ID NO: 9.

6. The method for treating kidney cancer or the method for preventing exacerbation of kidney cancer according to claim 1, wherein the sample is feces.

7. The method for treating kidney cancer or the method for preventing exacerbation of kidney cancer according to claim 1, wherein the nucleotide sequence information on the intestinal bacterium-derived genomic DNA is nucleotide sequence information on the 16S rRNA gene.

8. The method for treating kidney cancer or the method for preventing exacerbation of kidney cancer according to claim 1, wherein the test subject is a Japanese person.

9. The method for treating kidney cancer or the method for preventing exacerbation of kidney cancer according to claim 3, wherein the sample is feces.

10. The method for treating kidney cancer or the method for preventing exacerbation of kidney cancer according to claim 3, wherein the nucleotide sequence information on the intestinal bacterium-derived genomic DNA is nucleotide sequence information on the 16S rRNA gene.

11. The method for treating kidney cancer or the method for preventing exacerbation of kidney cancer according to claim 3, wherein the test subject is a Japanese person.

12. The method for treating kidney cancer or the method for preventing exacerbation of kidney cancer according to claim 4, wherein the bacterium of the genus Ruthenibacterium includes Ruthenibacterium lactatiformans having 16S rRNA gene having at least 97% sequence identity to the nucleotide sequence set forth in SEQ ID NO: 6, and a bacterium of the genus Ruthenibacterium having 16S rRNA gene having at least 97% sequence identity to the nucleotide sequence set forth in SEQ ID NO: 7.

13. The method for treating kidney cancer or the method for preventing exacerbation of kidney cancer according to claim 4, wherein the sample is feces.

14. The method for treating kidney cancer or the method for preventing exacerbation of kidney cancer according to claim 4, wherein the nucleotide sequence information on the intestinal bacterium-derived genomic DNA is nucleotide sequence information on the 16S rRNA gene.

15. The method for treating kidney cancer or the method for preventing exacerbation of kidney cancer according to claim 4, wherein the test subject is a Japanese person.
